(19) United States
(12) Patent Application Publication
(10) Pub. No.: US 2005/0070881 A1
(43) Pub. Date: Mar. 31, 2005

(54) Transition section for a catheter

(76) Inventors: Richard Gribbons, Athenry (IE); Ashish Varma, Galway (IE); Noel Coyle, Galway (IE)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/670,465

(22) Filed: Sep. 26, 2003

(51) Int. Cl. A61M 25/00

(52) U.S. Cl. 604/525

(57) ABSTRACT

A catheter having a proximal shaft defining a guidewire lumen and an inflation lumen. The inflation lumen is arcuate shaped and reinforced with a tube having an arcuate shaped cross-section or another reinforcing means. The catheter also has a distal shaft, with a greater flexibility than the proximal shaft. The catheter also has a transition section. A proximal end of the transition section communicates with the proximal shaft, and a distal end communicating with the distal shaft. The transition section has a gradually increased flexibility from its proximal end to its distal end. The transition section includes a transition means creating the increased flexibility.

### FIELD OF THE INVENTION

The present invention is directed to a catheter for use in intralumenal procedures within particularly tortuous vessels.

### BACKGROUND OF THE INVENTION

Cardiovascular disease, including atherosclerosis, is the leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA”. The objective in angioplasty is to enlarge the lumen of the affected coronary artery by radial hydraulic expansion. The procedure is accomplished by inflating a balloon of a balloon catheter within the narrowed lumen of the coronary artery.

In addition to PTCA, catheters are used for delivery of stents or grafts, therapeutic drugs (such as vaso-occlusion agents or tumor treatment drugs) and radiopaque agents for radiographic viewing. Other uses for such catheters are well known in the art.

The anatomy of coronary arteries varies widely from patient to patient. Often a patient's coronary arteries are irregularly shaped, highly tortuous and very narrow. The tortuous configuration of the arteries may present difficulties to the physician in proper placement of a guidewire, and advancement of a catheter to a treatment site. A highly tortuous coronary anatomy typically will present considerable resistance to advancement of the catheter over the guidewire.

Therefore, it is important for a catheter to be highly flexible. However, it is also important for a catheter shaft to be stiff enough to push the catheter into the vessel in a controlled manner from a position far away from the distalmost point of the catheter.

Conventional catheter shafts for PTCA and other procedures typically include a proximal shaft, a transition section and a distal shaft having a flexible distal tip. In particular, the catheters have a proximal shaft, which is generally rigid for increased pushability and a more flexible distal shaft with a flexible distal tip for curving around particularly tortuous vessels. Often the proximal shaft is made stiff by the insertion of a thin biocompatible tube, such as a stainless steel hypotube, into a lumen formed within the proximal shaft. The transition section is the portion of the catheter shaft between the stiffer proximal shaft and the more flexible distal shaft, which provides a transition in flexibility between the two portions.

With some types of catheter construction, when an increase in resistance occurs during a procedure there is a tendency for portions of the catheter to collapse, buckle axially or kink, particularly in an area where flexibility of the catheter shaft shifts dramatically. Consequently, the transition section is often an area where the flexibility of the catheter gradually transitions between the stiff proximal shaft and the flexible distal shaft. It is known in the art to create a more gradual flexibility transition by spiral cutting a distal end of the hypotubing used to create stiffness in the proximal shaft. Typically, the spiral cut is longitudinally spaced farther apart at the hypotube proximal end creating an area of flexibility, and longitudinally spaced closer together at the hypotube distal end creating an area of even greater flexibility.

In a typical PTCA procedure, it may be necessary to perform multiple dilatations, for example, using various sized balloons. In order to accomplish the multiple dilatations, the original catheter must be removed and a second catheter tracked to the treatment site. When catheter exchange is desired, it is advantageous to leave the guidewire in place while the first catheter is removed to properly track the second catheter.

Two types of catheters commonly used in angioplasty procedures are referred to as over-the-wire (OTW) catheters and rapid exchange (RX) catheters. A third type of catheter with preferred features of both OTW and RX catheters, which is sold under the trademarks MULTI-EXCHANGE, ZIPPER MX, ZIPPER, and/or MX, is discussed below. An OTW catheter's guidewire shaft runs the entire length of the catheter and is attached to, or enveloped within, an inflation shaft. Thus, the entire length of an OTW catheter is tracked over a guidewire during a PTCA procedure. A RX catheter, on the other hand, has a guidewire shaft that extends within only the distalmost portion of the catheter. Thus, during a PTCA procedure only the distalmost portion of a RX catheter is tracked over a guidewire.

If a catheter exchange is required while using a standard OTW catheter, the user must add an extension onto the proximal end of the guidewire to maintain control of the guidewire, slide the catheter off of the extended guidewire, slide the new catheter onto the guidewire and track back into position. Multiple operators are required to hold the extended guidewire in place while the original catheter is changed out.

A RX catheter avoids the need for multiple operators when changing out the catheter. With a rapid exchange catheter, the guidewire is outside the shaft of the catheter for all but the distalmost portion of the catheter. The guidewire can be held in place without an extension when the catheter is removed from the body. However, one problem associated with RX catheters is that the exposed portion of the guidewire may become tangled with the catheter shaft during use.

In addition, there are instances when the guidewire and not the catheter must be replaced. An OTW catheter, with the guidewire lumen extending the entire length of the catheter, allows for simple guidewire exchange. With a RX catheter, the guidewire, and most of the catheter, must be removed from the body in order to exchange guidewires. Essentially the procedure must then start anew because both the guidewire and the catheter must be retracked to the treatment site.

A balloon catheter capable of both fast and simple guidewire and catheter exchange is particularly advantageous. A catheter designed to address this need is sold by Medtronic AVE, Inc. of Santa Rosa, Calif. under the trademarks MULTI-EXCHANGE, ZIPPER MX, ZIPPER and/or MX (hereinafter referred to as the “MX catheter”). An MX catheter is disclosed in U.S. Pat. No. 4,988,356 to Crittenden et al., and in co-pending U.S. patent application Ser. No. 10/116,234, filed Apr. 4, 2002, both of which are incorporated in their entirety herein by reference.

The MX catheter includes a catheter shaft having a guidewire lumen positioned side-by-side with an inflation lumen. The MX catheter also includes a longitudinal cut that extends along the catheter shaft and that extends radially from the guidewire lumen to an exterior surface of a catheter shaft. A guide member through which the shaft is slidably coupled cooperates with the longitudinal cut such that a guidewire may extend transversely into or out of the guidewire lumen at any location along the longitudinal cut's length. By moving the shaft with respect to the guide member, the effective over-the-wire length of the MX catheter is adjustable.

The guidewire is threaded into a guidewire lumen opening at the distal end of the catheter and out through the guide member. The guidewire lumen envelops the guidewire as the catheter is advanced into the patient's vasculature. Furthermore, the indwelling catheter may be removed by withdrawing the catheter from the patient while holding the proximal end of the guidewire and the guide member in a fixed position. When the catheter has been withdrawn to the point where the distal end of the slot has reached the guide member, the distal portion of the catheter over the guidewire is of a sufficiently short length that the catheter may be drawn over the proximal end of the guidewire without releasing control of the guidewire or disturbing its position within the patient.

When both an inflation lumen and a guidewire lumen are generally circular in shape, a side-by-side lumen configuration generally creates a catheter shaft having an oblong or oval shaped cross-section. However, a catheter with an oblong shaped cross-section may not readily traverse the generally circular shaped body lumens as effectively as a catheter having a generally circular shaped cross-section.

### BRIEF SUMMARY OF THE INVENTION

In light of the foregoing discussed in the background section, the present invention is directed to a catheter shaft with a substantially circular cross-section having a rigid proximal shaft, a flexible distal shaft and a transition section, which gradually increases in flexibility from a proximal to a distal end thereof due to the inclusion of a transition means. The transition section has a proximal end and a distal end, such that the proximal end is in communication with the proximal shaft while the distal end is in communication with the distal shaft.

At least the proximal shaft defines a guidewire lumen and an inflation lumen. The inflation lumen is generally an arcuate shaped lumen (i.e., has an arcuate shaped cross-section) that cradles the guidewire lumen along the length of the proximal shaft. The proximal shaft also includes a reinforcing means. The reinforcing means provides increased pushability of the proximal shaft for controlling a distal portion of the catheter shaft from a proximal position. The reinforcing means may be an arcuate shaped tube inserted into the inflation lumen. Alternatively, the reinforcing means may be a rod, a long, thin plate or a skived or halved metal or thermoplastic tube inserted into the inflation lumen. Further, the reinforcing means may be entirely embedded in an extruded thickness of the proximal shaft.

Thus, the transition section is proximally defined where the stiffness of the reinforcement means ends or begins to be reduced and distally defined by the location of a transition means. For example, the transition section may contain a spiral helix as the transition means. The spiral helix may be disposed on the outside of the transition section or inside the transition section. Alternatively, the spiral helix may be bonded to the transition section, or may be positioned to cover more than one of the transition section, proximal shaft or distal shaft. The spiral helix may be partially bonded and partially free-floating. Alternatively, the spiral helix may be entirely free-floating within the transition section and held in place by a “bumped” reduction in the diameter between the transition section and the distal shaft. Also, the spiral helix may be extruded into the tubing of the transition section.

The transition means may be a continuation of the reinforcing means used in the proximal shaft, wherein the reinforcing means is skived or reduced as it extends distally from the proximal end to the distal end of the transition section. Thus, as the substance of the reinforcing means is reduced, the transition section becomes more flexible along its length.

If the catheter is an MX catheter, it has a longitudinal cut that is generally found between the guidewire lumen and an exterior surface of the proximal shaft. If the longitudinal cut continues into the transition section, any transition means located in or around the inflation lumen will not affect the distal movement of a guide member along the longitudinal cut. Thus, a skived or reduced reinforcing means within the inflation lumen will not affect this MX feature. However, a spiral helix located in or around the guidewire lumen may affect how far distally a guide member can move along a catheter shaft. Thus, the transition means in an MX catheter may be a U-shaped wire or ribbon sleeve that operates similarly to the spiral helix while providing an opening to access the longitudinal cut along the transition section. The U-shaped wire sleeve can be bent onto an exterior of transition section or it may be embedded into an extruded transition section.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art based on the teachings contained herein.

### BRIEF DESCRIPTION OF THE FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is a perspective view in partial cross-section of a catheter shaft according to an embodiment of the present invention.

FIG. 2 is a cross-section view of a proximal shaft of the present invention taken along line II-II of FIGS. 1, 5, 7, 8, 9 and 12.

FIGS. 3A and 3B are alternative cross-section views of a proximal shaft of the present invention taken along line II-II of FIGS. 1, 5, 7, 8, 9 and 12.

FIGS. 4A and 4B are alternative cross-section views of a proximal shaft of the present invention taken along line II-II of FIGS. 1, 5, 7, 8, 9 and 12.

FIG. 5 is a perspective view of a catheter shaft according to another embodiment of the present invention.

FIG. 6 is a cross-section view of a transition section of the present invention taken along line VI-VI of FIG. 5.

FIG. 7 is a perspective view in partial cross-section of a catheter shaft according to another embodiment of the present invention.

FIG. 8 is a perspective view in partial cross-section of a catheter shaft according to another embodiment of the present invention.

FIG. 9 is a perspective view in partial cross-section of a catheter shaft according to another embodiment of the present invention.

FIG. 10 is a cross-section view of a transition section of the present invention taken along line X-X of FIG. 9.

FIG. 11 is a cross-section view of a transition section of the present invention taken along line XI-XI of FIG. 9.

FIG. 12 is a perspective view in cross-section of a catheter shaft according to another embodiment of the present invention.

FIG. 13 is a cross-sectional view of a transition section of the present invention taken along a line XIII-XIII of FIG. 12.

FIG. 14 is a bent ribbon or wire used to form a U-shaped sleeve of the present invention.

FIG. 15 is a perspective view of the U-shaped sleeve of the present invention as seen in FIG. 10.

### DETAILED DESCRIPTION OF THE INVENTION

The present invention will be described with reference to the accompanying drawings. The drawing in which an element first appears is typically indicated by the leftmost digit(s) in the corresponding reference number.

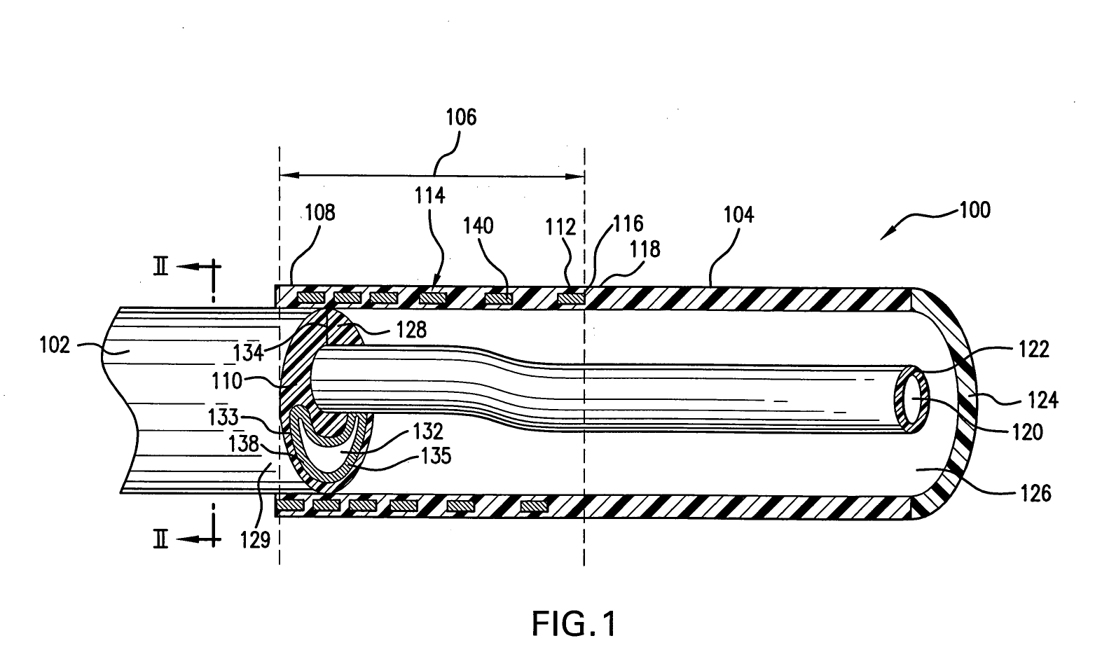

FIG. 1 shows a partial perspective view and partial cross-section of an embodiment of the present invention. In particular, FIG. 1 shows a catheter shaft 100 that includes a proximal shaft 102, a distal shaft 104 and a transition section 106. In this case, transition section 106 has a proximal end 108, which is defined by a distal end 110 of proximal shaft 102 and fluidly connected thereto. In the embodiment shown in FIG. 1, transition section 106 has a distal end 112 which is defined by a transition means 114, particularly by a distal end 116 of transition means 114. The distal end 112 of transition section 106 is fluidly connected to a proximal end 118 of distal shaft 104.

In the embodiment of FIG. 1, distal shaft 104 includes a coaxial guidewire lumen 120 defined by an inner shaft 122. Distal shaft 104 also includes an outer shaft 124, shown in FIG. 1 in partial cross-section. The area between outer shaft 124 and inner shaft 122 defines an inflation lumen 126. Proximal shaft 102 is made from a single extruded shaft 128 with a proximal guidewire lumen 230 and a proximal inflation lumen 132.

Outer shaft 124, inner shaft 120 and extruded shaft 128 are manufactured separately from thermoplastic materials, particularly high-density polyethylene, polyimide, polyamides, polyolefins and various other polymeric material. Preferably, outer shaft 124, inner shaft 120 and extruded shaft 128 are made from a polyethylene block amide (PEBA) copolymer. Outer shaft 124, inner shaft 120 and extruded shaft 128 are bonded together by an adhesive bond, a lap joint thermal compression bond, laser welding or ultrasonic welding or another method of bonding these thermoplastic materials together. Outer shaft 124 and inner shaft 120 may be extruded, molded or formed in another process known in the art for producing tubing used in a medical device.

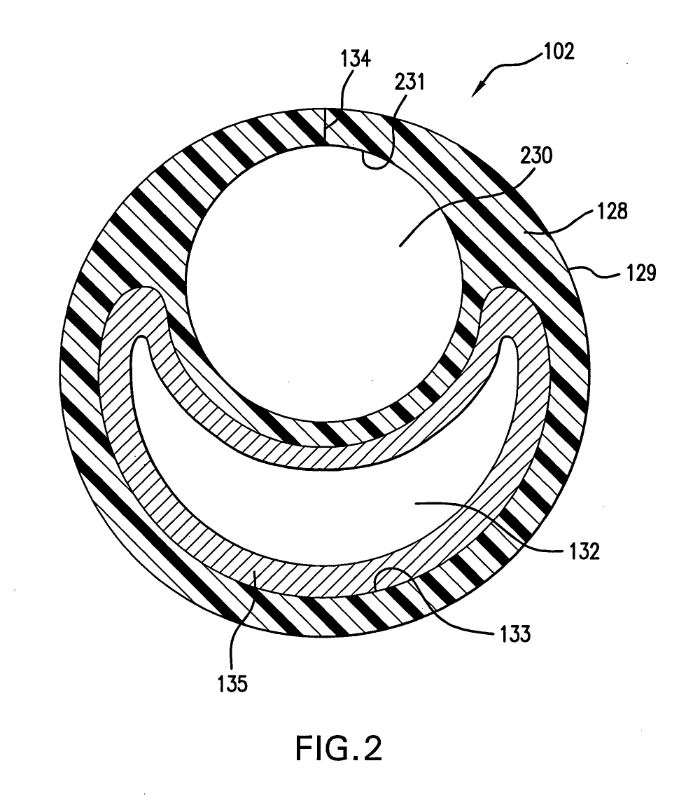

FIG. 1 and FIG. 2 show a cross-section of one embodiment of proximal shaft 102. FIG. 1 shows where proximal shaft 102 meets transition section 106. FIG. 2, however, shows a cross-section of proximal shaft along a line II-II of FIG. 1.

As seen in FIGS. 1 and 2, proximal shaft 102 includes extruded shaft 128 having a generally circular exterior surface 129 when viewed in cross-section in FIG. 2. Extruded shaft 128 defines a generally circular guidewire lumen 230 by a first interior surface 231. Extruded shaft 128 also defines arcuate shaped inflation lumen 132 by a second interior surface 133. The curved shape of inflation lumen 132 cradles guidewire lumen 230, so that proximal shaft 102 has an overall generally circular shaped cross-section.

One skilled in the art can appreciate that in an alternate embodiment a guidewire lumen may be arcuate shaped while an inflation lumen is generally circular. However, it is easier to track a guidewire through a circular shaped guidewire lumen rather than an arcuate shaped one. Inflation lumen 132 functions to fluidly communicate an inflation fluid with a balloon (not shown) at its distal end, so it may be of any shape, provided that enough volume of fluid can flow therethrough to inflate the balloon.

A side-by-side lumen arrangement, such as that shown in FIGS. 1 and 2, is particularly suited to an OTW or a MX catheter shaft type. The embodiment of proximal shaft 102 of FIG. 2 is an MX catheter shaft because it includes a longitudinal cut 134 through which a guidewire can exit guidewire lumen 230. An OTW catheter shaft is similar to FIG. 2, but without longitudinal cut 134. A catheter shaft 100 with a generally circular cross-section easily traverses a body lumen, which also has a generally circular shaped cross-section. Thus, an OTW or MX catheter having the structure of FIG. 2 may have a smaller profile and is easier to navigate through a body lumen than a conventional OTW or MX catheter having an oblong or oval cross-section.

A RX catheter has a single lumen proximal shaft, because it has a guidewire lumen only at a very distal portion of its catheter shaft 100. In a RX catheter, the cross-section shown in FIG. 2 could occur at the very distalmost portion of its proximal shaft or only in a distal or transition section.

One skilled in the art can appreciate how the transition section and various transition means of the present invention, described in detail below, may be suitable for a fixed wire, OTW, RX or MX catheter type.

In an embodiment of the present invention, proximal shaft 102 is reinforced with a reinforcing means. A reinforcing means provides proximal shaft 102 with increased pushability. In other words, the reinforcing means makes the proximal shaft 102 stiffer, so that a user can control the catheter while it traverses the tortuous pathways of the body lumen from a proximal position. In one embodiment, a reinforcing means is used along an entire length of the proximal shaft 102.

In conventional catheters, reinforcing means may be a thin metal tube, such as a stainless steel hypotube, inserted into a guidewire lumen or inflation lumen in order to reinforce the proximal shaft. An MX catheter design, however, is not suitable for having a hypotube inserted within guidewire lumen 230 because the guidewire must be able to escape out of longitudinal cut 134 made between the guidewire lumen and exterior surface 129 of extruded shaft 128 as shown in FIG. 2. Meanwhile, a hypotube is not suitable for use in the inflation lumen shown in FIG. 2 because the inflation lumen is arcuate shaped rather than circular. Thus, an embodiment of the present invention must have a different type of reinforcing means.

FIG. 2 shows an arcuate shaped reinforcing means 135. Arcuate shaped reinforcing means 135 may be a tubing that is cast in the particular arcuate shape or it may be a thin tube, such as a hypotube, which has been crimped to form the arcuate shape. Alternatively, reinforcing means 135 may be a plastic material having a high rigidity.

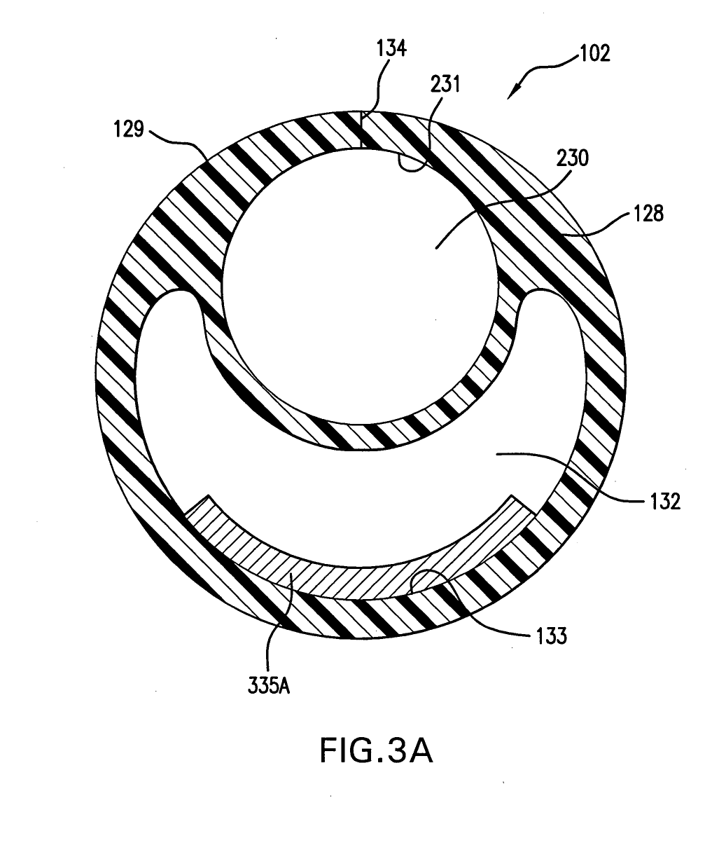

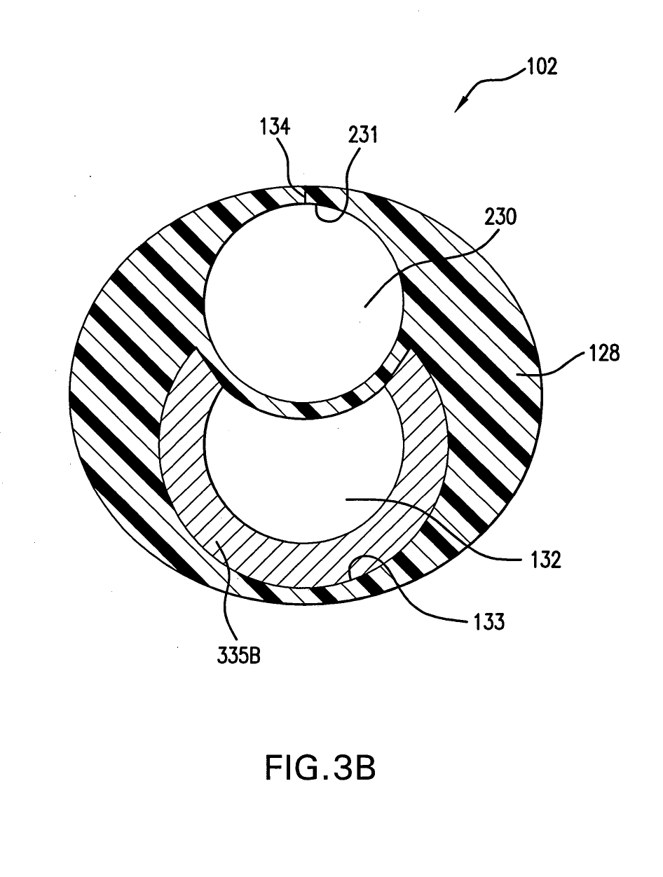

FIG. 3A and FIG. 3B show further embodiments of a reinforcing means. Reinforcing means 335A and 335B may be a metal or thermoplastic plate or rod in a flat, curved or cylindrical shape. If the reinforcing means 335A or 335B are curved, they may be pressed into shape, cut from a hypotube, or extruded into a curved shape. The advantage of having a reinforcing means other than a hypotube is that a rod or plate, such as that shown in FIG. 3A, takes up less room inside inflation lumen 132, thereby allowing for a greater volume of inflation fluid to pass therethrough. Further, without the double walls of a tubular reinforcing means, the overall dimensions of catheter shaft 100 may be reduced, such that the catheter shaft will then have a lower profile. The type of reinforcing means shown in FIG. 3A could also be inserted into the guidewire lumen of a generally circular catheter shaft 100, provided that it does not interfere with a guidewire's movement through a guidewire lumen or its exit through longitudinal cut 134 of an MX catheter type.

FIG. 3B shows a slightly different arcuate shaped inflation lumen 132 including a hypotube reinforcing means 335B, in which a top portion has been skived off of the hypotube. Various other shapes of inflation lumen 132 similarly reinforced would be appropriate for use in this invention.

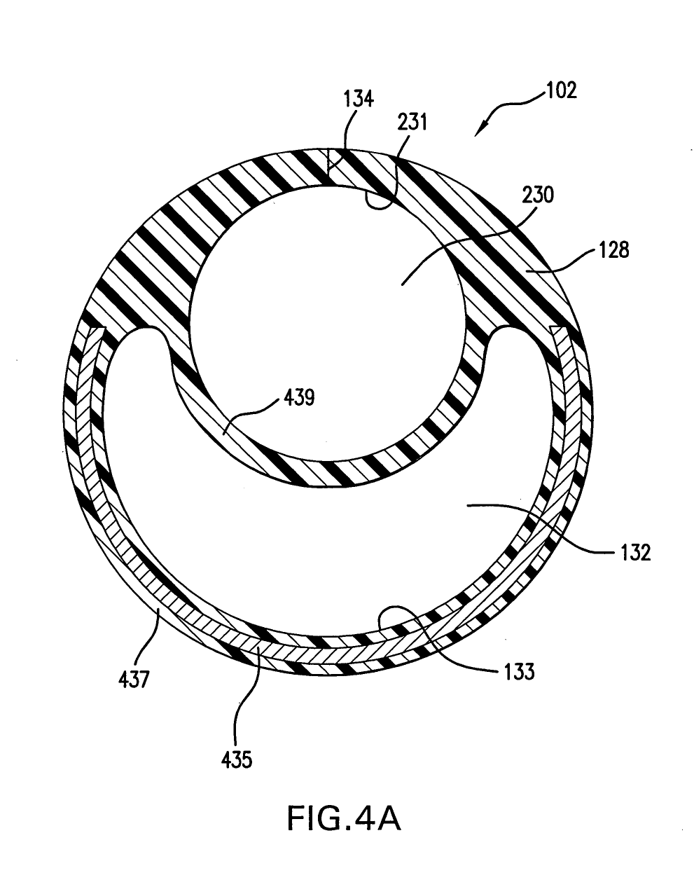

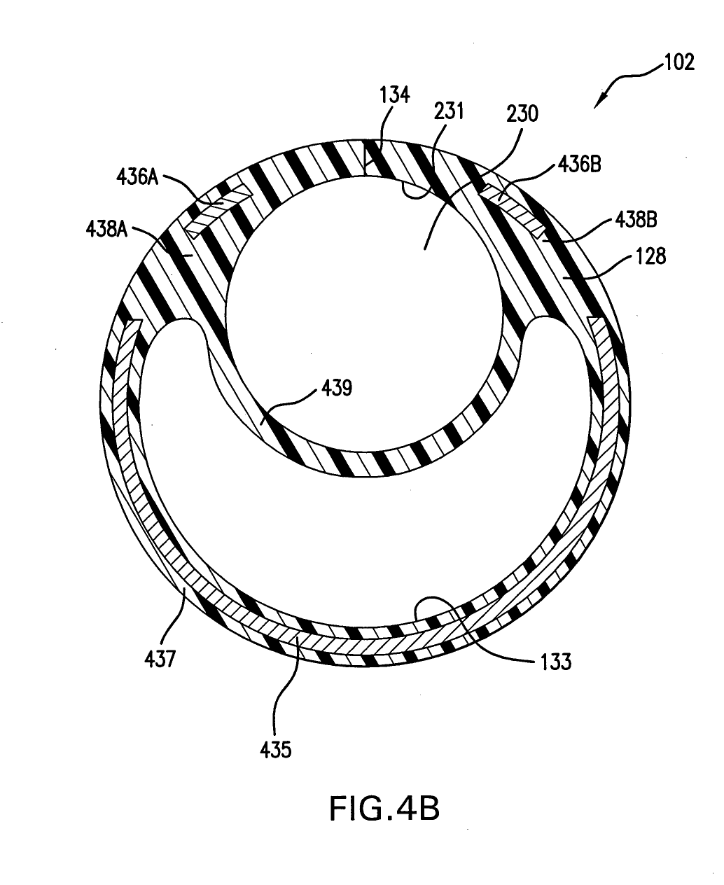

In another embodiment, seen in FIG. 4A and FIG. 4B, a reinforcing means 435 may be embedded into the extruded shaft 128. In FIG. 4A, reinforcing means 435 is a half tube, such as half of a stainless steel hypotube, which has been extruded into a portion 437 of extruded shaft 128 on the opposite side of inflation lumen 132 from guidewire lumen 230. Alternatively, a reinforcing means may be extruded into another portion of extruded shaft 128. For example, reinforcing means may be located at portion 439 between guidewire lumen 230 and inflation lumen 132. Also, as shown in FIG. 4B, support strips 436A and 436B may be placed in another location 438A and 438B just adjacent to guidewire lumen 230. Support strips 436A and 436B may be extruded along with reinforcing means 435, or as an alternative thereto. Further, only one or the other of support strips 436A and 436B may be embedded into extruded shaft 128. Other catheter designs such as the coaxial OTW, fixed wire and RX catheters may also have a reinforcing means extruded into the extruded shaft 128 of the proximal shaft 102.

FIGS. 2, 3A, 3B, 4A and 4B provided only a few ways in which proximal shaft may be reinforced in accordance with the present invention. The present invention may be suitable for use with other reinforcing techniques.

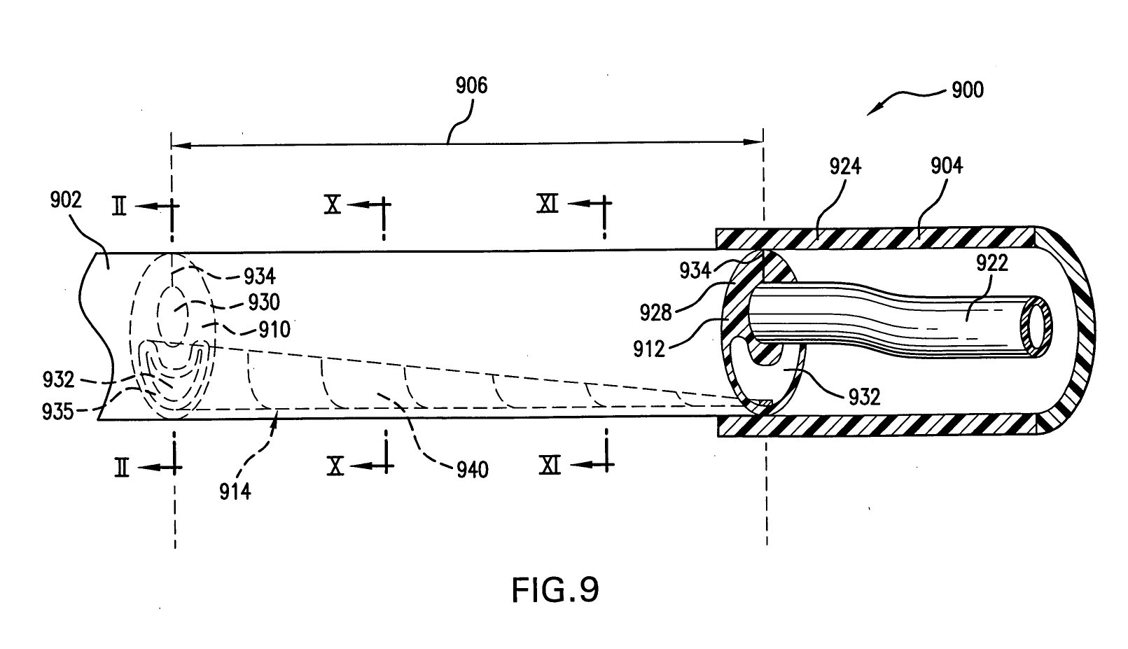

In FIG. 1, the coaxial structure of distal shaft 104 extends into transition section 106, such that extruded shaft 128 is inserted and bonded inside outer shaft 124. Similarly, inner shaft 122 is inserted into and bonded to proximal guidewire lumen 230 of proximal shaft 102. In an alternate embodiment, however, extruded shaft 128 of proximal shaft 102 may extend distally into transition section 106 without the additional support provided by reinforcing means 135. For example, FIG. 9 shows such a transition section, as will be discussed in detail below.

As seen in to FIG. 1, distal end 110 of proximal shaft 102 occurs simultaneously with a distal end 138 of reinforcing means 135. The distalmost end of catheter shaft 100 must be highly flexible to curve around the most tortuous parts of the vasculature. However, an abrupt end to the stiffness created by reinforcing means 135, of FIG. 1, may cause a procedurally disastrous kink in catheter shaft 100. Thus, transition section 106 extends from distal end 138 of reinforcing means 135 to distal end 116 of transition means 114 to provide a transition between the rigidity of proximal shaft 102 and the flexibility of distal shaft 104.

In a conventional catheter shaft, a transition means may be created by spiral cutting the reinforcing means. However, since the reinforcing means in this case is not a circular hypotube, the present invention provides transition means alternative to spiral cutting a reinforcing means.

In FIG. 1, transition means 114 is a spiral helix 140. Spiral helix 140 may be made of a metal wire or ribbon twisted to form a coil. Alternatively, the spiral helix 140 may be made from a thermoplastic polymer having sufficient strength to provide support to transition section 106. Preferably, spiral helix 140 is a wire ribbon, which will lay flat, such that it may be embedded into outer shaft 124 upon extrusion thereof without significantly increasing the outer diameter of outer shaft 124. Having spiral helix 140 embedded into an extruded outer shaft 124 allows for easier assembly of catheter shaft 100 due to fewer individual components. In addition, it retains a smooth outer wall surface to aid in moving through a body lumen.

Spiral helix 140 provides a gradual increase in flexibility by having the pitch of the coils closer together at proximal end 108 and further apart at distal end 116 of transition section 106. Further, moving distally along spiral helix 140 the windings become farther apart. Where the windings of the coil are closer together, the spiral helix 140 has less movement, thus making the transition section 106 stiffer. However, where the coils are farther apart, the spiral helix 140 has more movement and more flexibility. Therefore, the spiral helix 140 provides a gradual transition in flexibility along transition section 106. In addition, spiral helix 140 may be of any length and the pitch may be altered such that a desired flexibility occurs at a particular location along transition section 106.

In an alternate embodiment, extruded shaft 128 may extend into transition section 106 without reinforcing means 135. Thus, transition means 114 may be disposed in outer surface 124 in a location where extruded shaft 128 and outer shaft 124 overlap. Alternatively, spiral helix 140 may be extruded into extruded shaft 128 at a position distal to the distal end 138 of reinforcing means 135.

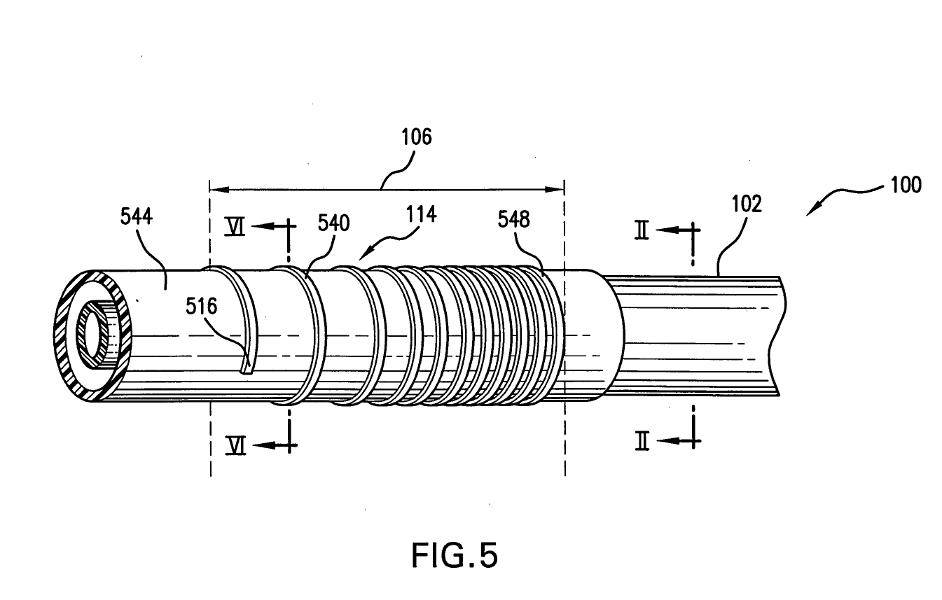

FIG. 5 shows an exterior perspective view of an alternate embodiment of the present invention. FIG. 5 includes a proximal shaft 102, a distal shaft 104 and a transition section 106, as discussed above for FIG. 1. For example, proximal shaft 102 may have a cross-section along line II-II, which takes the form of any of the cross-sections shown in FIGS. 2-4 or may include another type of reinforcing means.

However, FIG. 5 has a spiral helix 540 as transition means 114 positioned on an outer surface 544 of outer shaft 124 rather than embedded therein. Again, spiral helix 540 may be a coiled ribbon or wire, but is preferably a ribbon, which lays flat against the outer surface 544 of outer shaft 124.

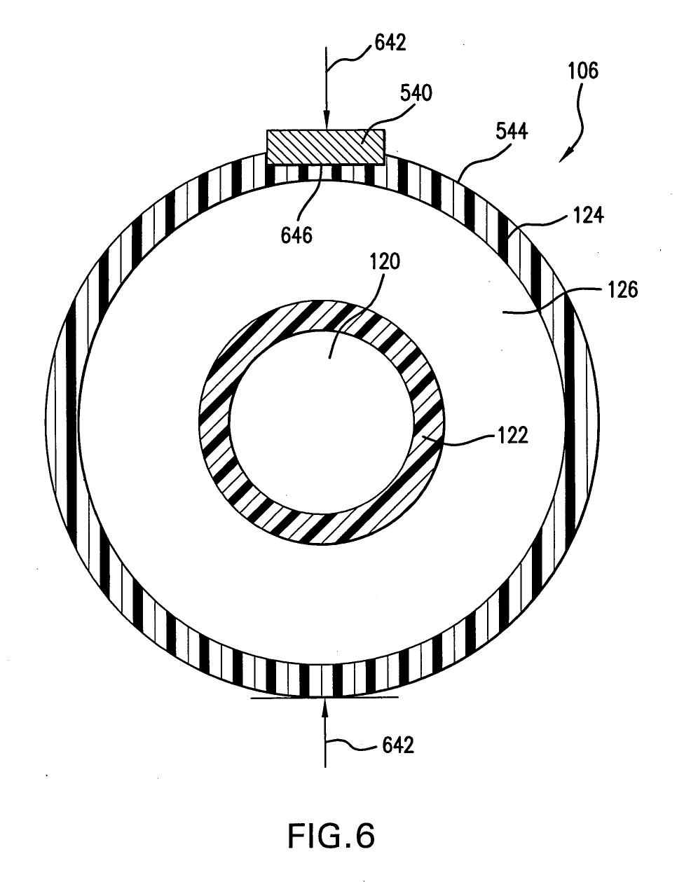

FIG. 6 shows a cross-sectional view of transition section 106 at a line VI-VI of FIG. 5. FIG. 6 shows transition section 106 having inner shaft 122 defining guidewire lumen 120. Transition section 106 also has inflation lumen 126 defined by the area between inner shaft 122 and outer shaft 124. FIG. 6 shows how a ribbon spiral helix 540 creates a small outer diameter 642, by remaining somewhat flush to outer surface 544. A wire spiral helix 540 would have a round cross-section rather than the generally rectangular cross-section shown in FIG. 6. FIG. 6 also shows how outer surface 544 of outer shaft 124 may have an indentation 646, which receives spiral helix 540 to create an even smaller outer diameter 642. A laser may accurately draw indentation 646 onto outer surface 544 or indention 646 may be imprinted onto a soft polymer surface. Spiral helix 540 may be secured to the outer surface 544 along the entire length of spiral helix 540 by adhesive bonding, heat fusion, laser bonding, an interference fit or another type of bonding.

Alternatively, spiral helix 540 may be fully or partially free-floating along outer surface 544 of outer shaft 124. As such only a portion or an end of spiral helix 540 would be bonded to outer surface 544 of outer shaft 124. A fully or partially free-floating spiral helix 540 may provide greater flexibility for transition section 106, but may cause greater friction against the walls of a body lumen when inserted therein. Alternatively, spiral helix 540 may be placed between outer surface 544 and a thin coating or covering, such as a layer of polyolefin, polyimide or polyamide, to reduce friction when moving through a body lumen and to hold spiral helix 540 in place.

Spiral helix 540 may vary in pitch (i.e. distance between adjacent windings) from a proximal end 548 to a distal end 516. In particular, near the proximal end 548, spiral helix 540 has a tight pitch, wherein the windings are close together. At the distal end 516, spiral helix 540 has a looser pitch, wherein the windings are farther apart. Thus, from proximal end 548 to distal end 516, the flexibility of spiral helix 540 increases with an increase in the pitch of the coils, providing transition section 106 with a gradual increase in flexibility.

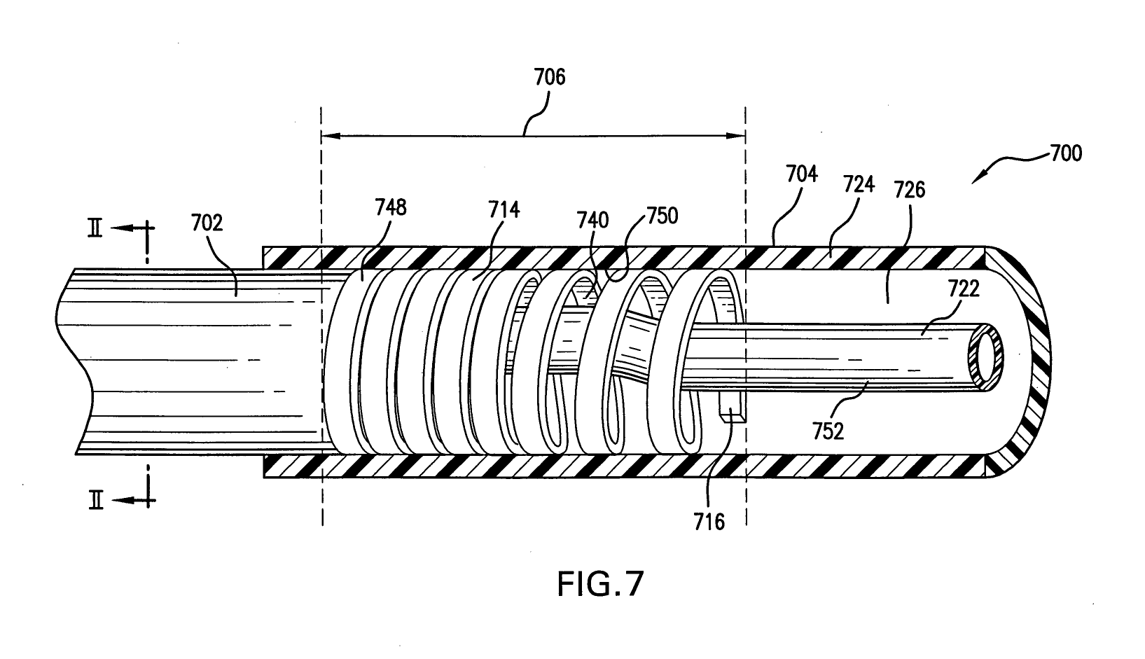

FIG. 7 shows another embodiment of the present invention. FIG. 7 shows a catheter shaft 700 similar to catheter shaft 100 of FIG. 1, with a proximal shaft 702, distal shaft 704 and transition section 706. Proximal shaft 702 may have a cross-section along line II-II, which takes the form of any of the cross-sections shown in FIGS. 2-4 or may include another type of reinforcing means. In the embodiment of FIG. 7, transition means 714 is a spiral helix 740 positioned between outer shaft 724 and inner shaft 722. In other words, spiral helix 740 is located within inflation lumen 726.

Spiral helix 740 may be bonded at a proximal end 748 to proximal shaft 702. If so, a distal end 716 of spiral helix 740 may be free-floating inside outer shaft 724. Alternatively, spiral helix 740 may be bonded to an interior surface 750 of outer shaft 724 at one or more locations or along the entire length of spiral helix 740. For example, the distal end 716 of the spiral helix 740 may be bonded to the outer shaft 724, and the proximal end 748 may be free-floating. Spiral helix 740 also may be located between outer shaft 724 and a coating or covering used to keep spiral helix 740 in position and to isolate spiral helix 740 from the inflation fluid flow. In yet another embodiment, a spiral helix may be used that has smaller outer diameter than the spiral helix 740 shown in FIG. 7, such that it lays flat against an outer surface 752 of inner shaft 722, in a similar fashion to spiral helix 540 which lies against outer shaft 124 as shown in FIGS. 5 and 6.

As discussed above, from proximal end 748 to distal end 716, the flexibility of spiral helix 740 increases with an increase in the pitch between the windings, providing transition section 706 with a gradual increase in flexibility.

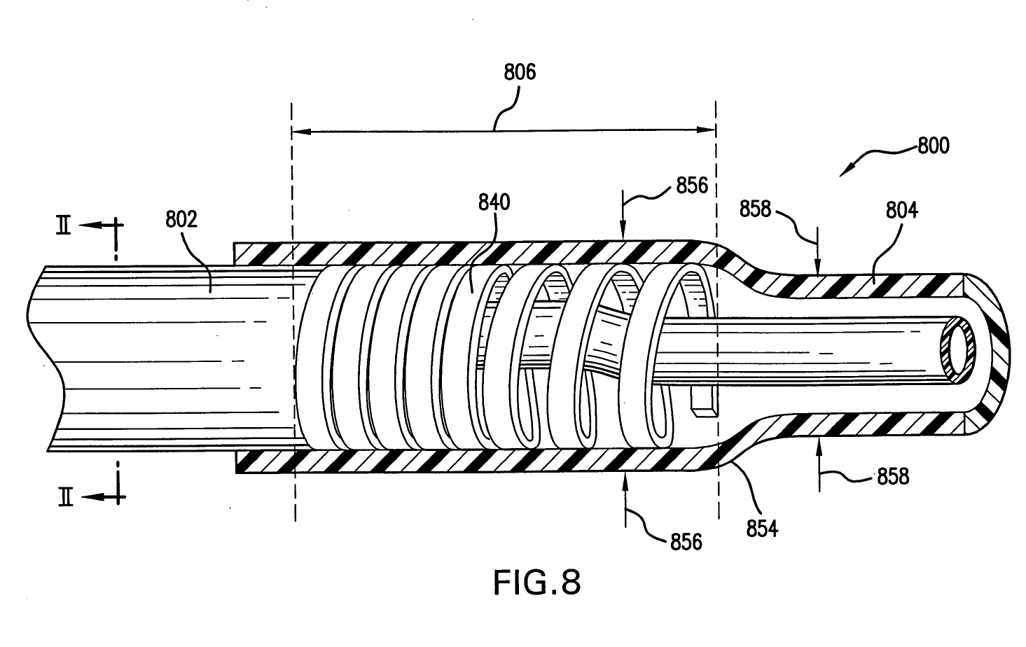

Again, a spiral helix that is free floating will provide the greatest flexibility. However, spiral helix 740 of FIG. 7, if not bonded to any part of proximal shaft 702 or transition section 706, may move proximally or distally within outer shaft 724. FIG. 8, however, shows another embodiment of the present invention, identical to FIG. 7, except that outer shaft 824 has a “bumped” region 854 wherein the diameter of outer shaft 824 reduces from a first diameter 856 to a second diameter 858. The second diameter 858 is less than the diameter of the spiral helix 840, thus preventing spiral helix 840 from shifting proximally and distally inside of outer shaft 824. In the catheter shaft 800 of FIG. 8, spiral helix 840 can float freely for maximum flexibility.

If a catheter shaft of the previously described embodiments, such as catheter shaft 100 as shown in FIG. 1, is utilized in a MX catheter design, then the longitudinal cut 134 in the proximal shaft 102 will only be accessible up to the spiral helix 140, because the spiral helix forms a closed loop around either the interior or the exterior of the transition section 106. Thus, a guide member of an MX catheter will not be able to open longitudinal cut 134 at a location of or distal to the spiral helix 140. This is true for the spiral helix of each of the previously described embodiments.

With reference to FIG. 1, the guide member (not shown) of an MX catheter opens longitudinal cut 134 and leads the guidewire out of guidewire lumen 230. However, in order that catheter shaft 100 is easily exchanged, the guide member must move distally far enough along the catheter shaft 100 that the operator can reach the guidewire distally of catheter shaft 100 while holding a proximal portion of the guidewire. Thus, distal end 110 of proximal shaft 102 (i.e., where longitudinal cut 134 ends) must be sufficiently close to the distal end of catheter shaft 100 for the MX catheter function to operate. However, a closed loop spiral helix as a transition means, as described in each of the previous embodiments, is well suited for use at any location with OTW, fixed wire and most RX catheters.

FIG. 9 shows another embodiment of the present invention. Catheter shaft 900 of FIG. 9 also includes a proximal shaft 902, a distal shaft 904 and a transition section 906. Also proximal shaft 902 may have a cross-section along line II-II that takes the form of any of the cross-sections shown in FIGS. 2-4 or may include another type of reinforcing means. In this case, extruded shaft 928 extends into transition section 906. In this embodiment, a transition means 914 is essentially a portion 940 of a reinforcing means 935 in which part of the structure of the reinforcing means is gradually removed along the length of transition section 906. For example, in FIG. 9, a proximal portion of reinforcing means 935 is an arcuate shaped tube, such as is shown in FIG. 2. However, distally of the distal end 910 of proximal shaft 902 the reinforcing means 935 becomes reduced in size or shape to be thinner and more flexible. In this case, reinforcing means 935 may be skived away to almost nothing at a distal end 912 of transition section 906. As the reinforcing means 935 is reduced, the stiffness it provided to the proximal shaft 902 is reduced. Thus, the transition section 906 gradually becomes more flexible.

The shape and thickness of reinforcing means 935 may be changed in a variety of ways along the reduced portion 940 of reinforcing means 935 in order to achieve the desired flexibility at any location along the length of transition section 906. For example, if reinforcing means 935 is a rod or a metal plate, the rod or metal plate may be made thinner and more flexible at a distal end than at the proximal end where it is used as a reinforcing means.

Alternatively, the properties of reinforcing means 935 may be altered in portion 940, without changing the physical dimensions of the reinforcing means 935. For example, reinforcing means 935 may be made from a thermoplastic polymer having a particular stiffness in proximal shaft 902. However, in transition section 906 the properties of that polymer can be chemically altered to provide a gradual increase in flexibility along transition section 906. For example, transition section 906 may comprise two materials having different stiffness compositions, such that the concentration of each material (i.e., the percent composition) changes along the length of transition section 906 to provided different characteristics. Further, chemical processing, such as cross-linking, may also change the properties along transition section 906.

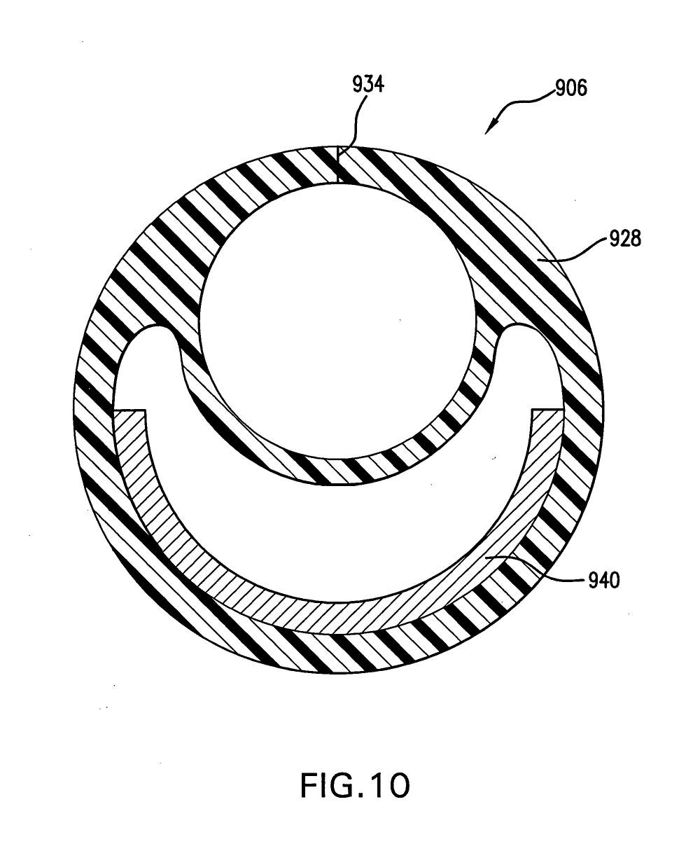

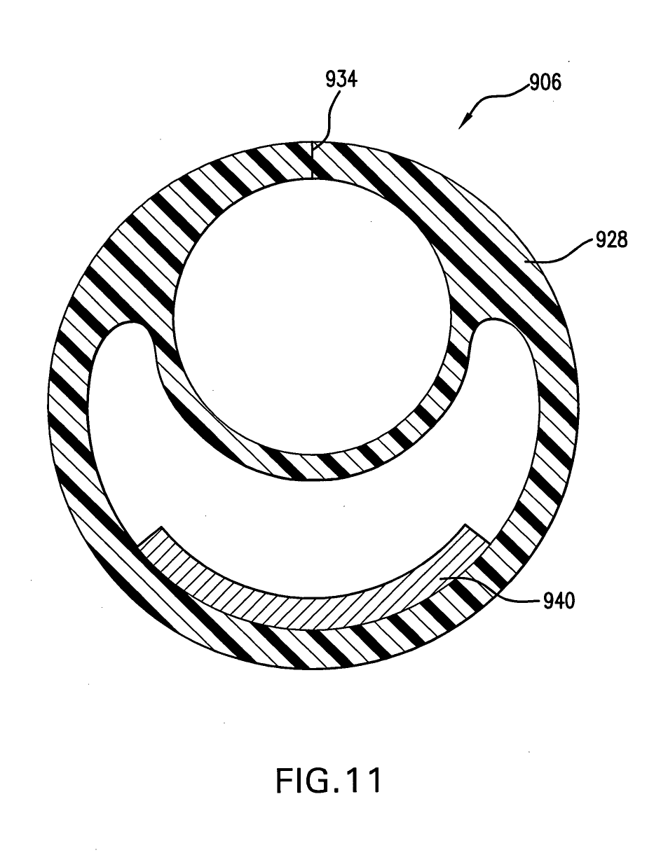

FIGS. 10 and 11 are cross-section views of transition section 906 taken along lines X-X and XI-XI, respectively. FIGS. 10 and 11 show a gradual reduction in the portion 940 of reinforcing means 935, which is shown in phantom in FIG. 9.

The embodiment of FIG. 9 shows that a longitudinal cut 934 may be used to access a guidewire in guidewire lumen 930 along the length of both the proximal shaft 902 and the transition section 906, at least up to where outer shaft 924 overlaps extruded shaft 928. Thus, a guide member (not shown) may be slid distally along longitudinal cut 934 of proximal shaft 902 until the guide member essentially reaches distal shaft 924. With such a manipulation, the guide wire effectively reduces a distal portion of guidewire lumen 930 so that the guidewire is accessible proximally at the guide member and distally at a distal tip of catheter 900 thereby allowing a single operator catheter exchange. A flat or curved reinforcing member placed anywhere in inflation lumen 932, guidewire lumen 930 or within extruded shaft 928, with the exception of certain locations near longitudinal cut 934, would provide access for a guide member to longitudinal cut 934.

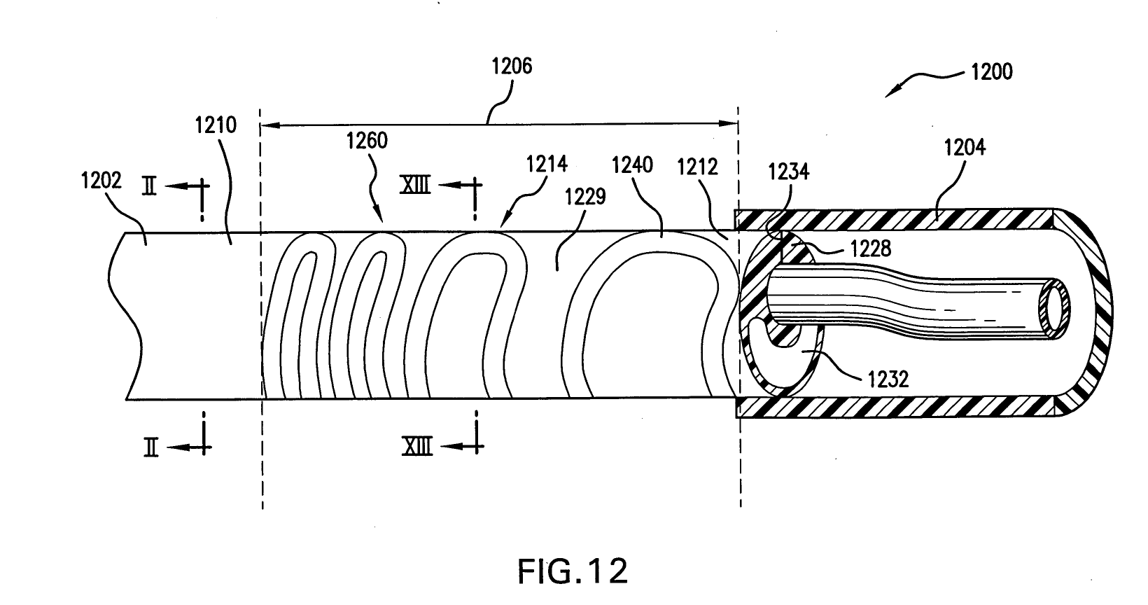

FIG. 12 shows yet another embodiment of the present invention. In this embodiment, catheter shaft 1200 includes a proximal shaft 1202, a distal shaft 1204 and a transition section 1206. In this case, extruded shaft 1228 extends into transition section 1206, similar to that of FIG. 9. However, reinforcing means 1235 does not extend distally beyond the distal end 1210 of proximal shaft 1202. Thus, guidewire lumen 1230, inflation lumen 1232 and/or extruded shaft 1228 do not have any reinforcing means therein within transition section 1206. In FIG. 12, transition means 1214 is a U-shaped sleeve 1240. U-shaped sleeve 1240 in FIG. 12 is located on the exterior surface 1229 of extruded shaft 1228. U-shaped sleeve has the advantages of the spiral helix described above but does not wrap the entire way around catheter shaft 1200. Thus, the U-shaped sleeve 1240 provides an opening 1260, such that longitudinal cut 1234 may be opened by a guide member up to a position near the distal end 1212 of transition section 1206.

U-shaped sleeve 1240 may be bonded to the outer surface 1229 of extruded shaft 1228 at any location or along the entire length of U-shaped sleeve 1240 by adhesive bonding, heat bonding, laser bonding or another type of bonding. Similarly to that of a spiral helix, a free-floating U-shaped sleeve 1240 may provide greater flexibility for transition section 1206, but will likely cause greater friction against the walls of a body lumen when inserted therein. U-shaped sleeve 1240 may be place between outer surface 1229 and a thin coating or covering, such as a layer of polyolefin or polyimide, to reduce friction when moving through a body lumen and to hold U-shaped sleeve 1240 in place. Alternatively, U-shaped sleeve 1240 may be extruded into extruded shaft 1228.

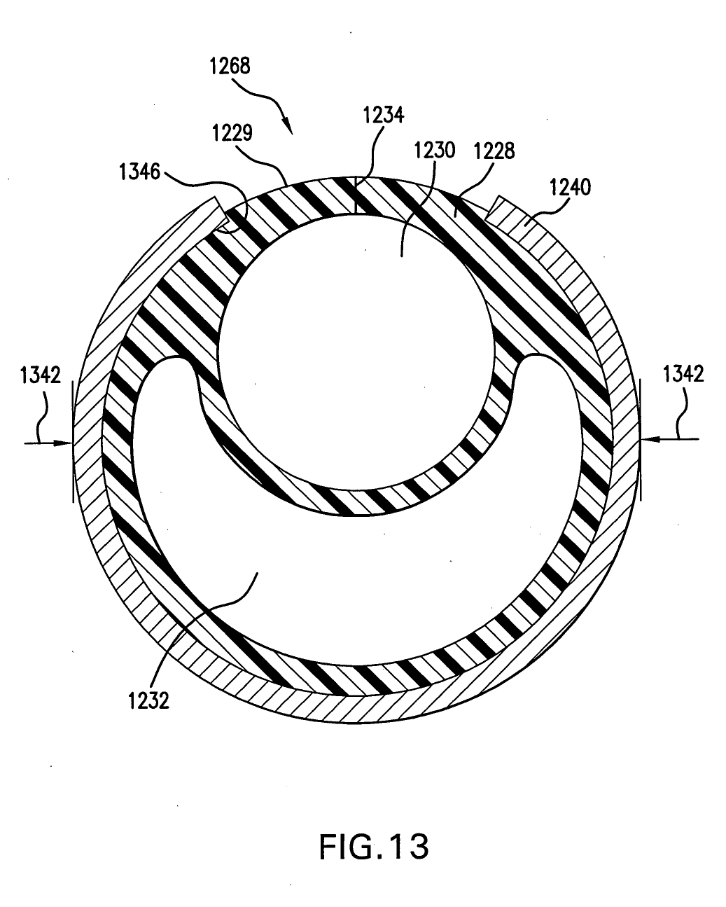

FIG. 13 shows a cross-sectional view of transition section 1206 at a line XIII-XIII of FIG. 12. FIG. 13 shows extruded shaft 1228 defining guidewire lumen 1230 and inflation lumen 1232. FIG. 13 shows how a U-shaped sleeve 1240 creates a small outer diameter 1342, by remaining somewhat flush to an outer surface 1229. FIG. 13 also shows how outer surface 1229 of extruded shaft 1228 may have an indentation 1346, which receives U-shaped sleeve 1240 to create an even smaller outer diameter 1342. FIG. 13 also shows opening 1268 in U-shaped member 1240, through which a guide member may travel to open longitudinal cut 1234 and release a guidewire placed within guidewire lumen 1230.

U-shaped sleeve 1240 may be formed from a ribbon or a wire. Alternatively, U-shaped sleeve 1240 may be made from a thermoplastic polymer having sufficient strength to provide support to transition section 1206. Preferably, U-shaped sleeve 1240 is a wire ribbon. As shown in FIG. 13, a ribbon, having a flatter cross-section than a rounded cross-section of a wire, may provide a lower outer diameter 1342 for catheter shaft 1200.

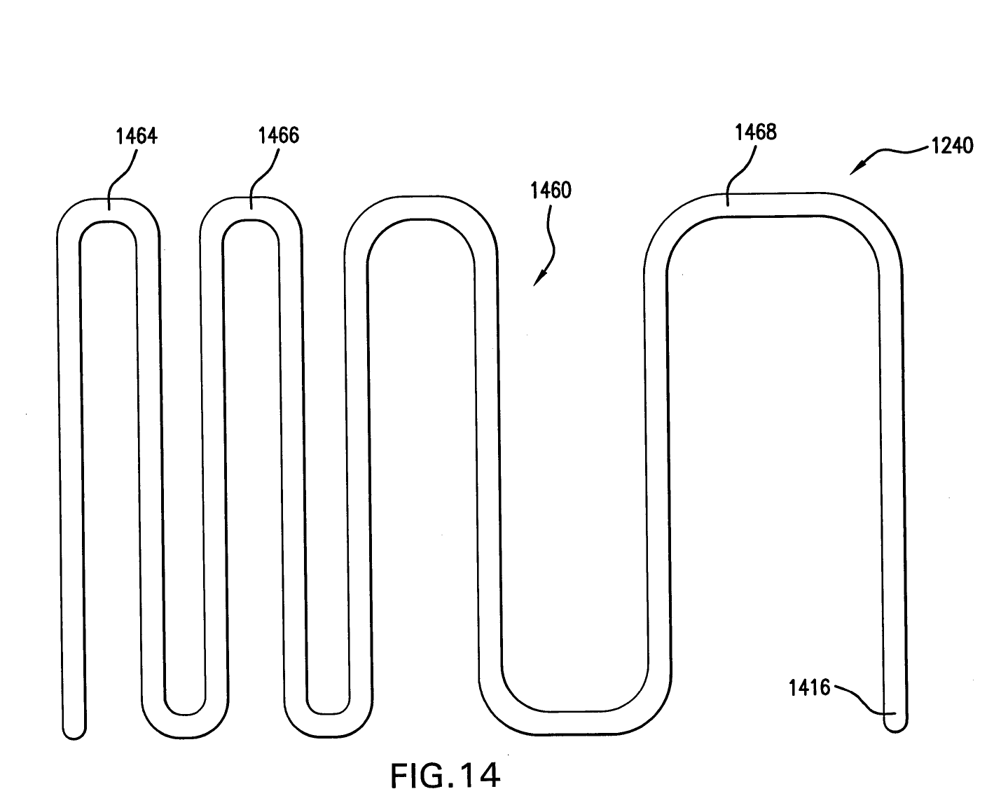

FIG. 14 shows how a ribbon or wire 1460 may be bent into a repeating series of loops 1464 having a generally sinusoidal or zigzag shape. The loops 1464 may be shaped as shown in FIG. 14, where the pitch between loops 1464 and the size of loops 1464 increases as you move along the ribbon 1460. Smaller loop 1466 will not provide transition section 1206 as much flexibility as will larger loop 1468. Thus, larger loop 1468 is closer to a distal end 1416 of U-shaped sleeve 1240, where greater flexibility is required. The ribbon 1460 of FIG. 14 may be formed by bending a ribbon or wire on a form tool. Alternatively, the ribbon or wire 1460 may be stamped out of a metal or plastic sheet.

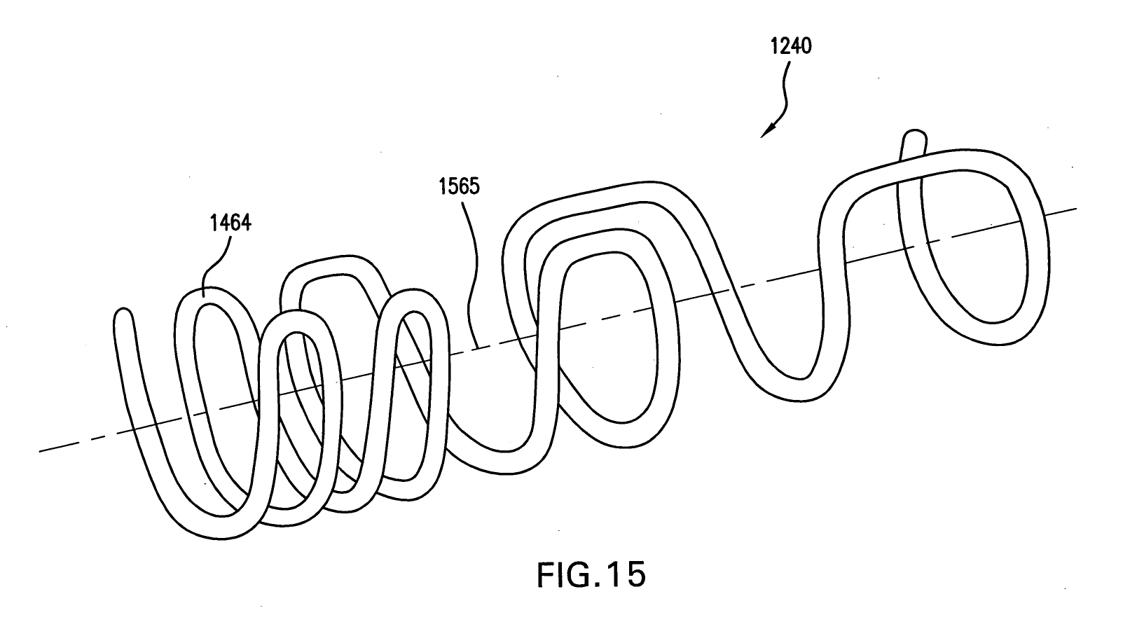

FIG. 15 shows how the ribbon or wire 1460 of FIG. 14 is bent to form U-shaped sleeve 1240. U-shaped sleeve 1240 generally curves around an axis 1565, which is a center point of the generally circular catheter shaft 1200. Loops 1464 are bent up such that opposite ends of loops in FIG. 14 face the same direction or face each other in FIG. 15, depending upon how far U-shaped sleeve is curved. U-shaped sleeve 1240 may be slid onto catheter shaft 1200 and crimped onto outer surface 1229 of extruded shaft 1228 of transition section 1206, as seen in FIG. 13. Alternatively, U-shaped sleeve 1240 may be bent onto extruded shaft 1228 directly from the ribbon or wire shape 1460 of FIG. 14.

While the invention has been particularly shown and described with reference to preferred embodiments thereof, it will be understood by those skilled in the art that they have been presented by way of example only, and not limitation, and various changes in form and details can be made therein without departing from the spirit and scope of the invention.

Thus, the breadth and scope of the present invention should not be limited by any of the above-described exemplary embodiments, but should be defined only in accordance with the following claims and their equivalents. Additionally, all references cited herein, including issued U.S. patents, or any other references, are each entirely incorporated by reference herein, including all data, tables, figures, and text presented in the cited references.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1. A catheter, comprising:
a proximal shaft defining a guidewire lumen and an inflation lumen, wherein said inflation lumen is arcuate shaped and reinforced with a tube having an arcuate shaped cross-section;
a distal shaft wherein said distal shaft has a greater flexibility than said proximal shaft; and
a transition section having a proximal end and a distal end, said proximal end communicating with said proximal shaft and said distal end communicating with said distal shaft, wherein said transition section has a gradually increased flexibility from said proximal to said distal ends.

2. The catheter of claim 1, wherein said gradually increased flexibility is created by a spiral helix in the transition section.

3. The catheter of claim 2, wherein said spiral helix is positioned on an exterior surface of said transition section.

4. The catheter of claim 2, wherein said spiral helix is positioned against an interior surface of said transition section.

5. The catheter of claim 2, wherein said spiral helix is entirely embedded into an extruded thickness of said transition section.

6. The catheter of claim 2, wherein subsequent windings of said spiral helix are closer together at said proximal end of said transition section and farther apart at said distal end of said transition section.

7. The catheter of claim 2, wherein said spiral helix is made from one of a metal ribbon or metal wire.

8. The catheter of claim 2, wherein said spiral helix is made from a thermoplastic polymer.

9. The catheter of claim 1, wherein said tube includes a skived portion at its distal end that extends within said transition section to achieve said gradually increased flexibility.

10. The catheter of claim 1, wherein said proximal shaft has a longitudinal cut along its length, said longitudinal cut extending in a radial direction between an exterior surface of said proximal shaft and said guidewire lumen.

11. The catheter of claim 10, wherein said longitudinal cut extends distally into said transition section.

12. The catheter of claim 11, wherein said gradually increased flexibility is created by a U-shaped wire sleeve, said wire sleeve having an opening for generally aligning with said longitudinal cut.

13. The catheter of claim 12, wherein said sleeve is a wire formed into a zigzag shape and bent into a generally curved shape.

14. The catheter of claim 13, wherein said sleeve is positioned around an exterior surface of said transition section.

15. The catheter of claim 13, wherein said sleeve is embedded within an extruded thickness of said transition section.

16. The catheter of claim 1, wherein said tube is made from one of a metal and a thermoplastic polymer.

17. A catheter, comprising:
a proximal shaft defining a guidewire lumen and an inflation lumen, wherein said inflation lumen is arcuate shaped and reinforced with a reinforcing means;
a distal shaft wherein said distal shaft has a greater flexibility than said proximal shaft; and
a transition section having a proximal end and a distal end, said proximal end communicating with said proximal shaft and said distal end communicating with said distal shaft, wherein said transition section includes a transition means embedded entirely within an extruded thickness of said transition section.

18. The catheter of claim 17, wherein said reinforcing means is a metal tube having an arcuate cross-section.

19. The catheter of claim 17, wherein said reinforcing means is one of a metal rod, a long thin metal plate, a skived metal tube and a halved metal tube.

20. The catheter of claim 17, wherein said reinforcing means is entirely embedded within an extruded thickness of said proximal shaft.

21. The catheter of claim 17, wherein said reinforcing means is disposed within an interior surface of said proximal shaft defining said inflation lumen.

22. The catheter of claim 17, wherein said transition means is a spiral helix.

23. The catheter of claim 22, wherein said spiral helix has a tight pitch at a first end and a loose pitch at a second end.

24. The catheter of claim 17, wherein said transition means is a portion of said reinforcing means.

25. The catheter of claim 24, wherein said transition means is a skived portion of a tube.

26. The catheter of claim 24, wherein said transition means is a rod that becomes thinner and more flexible along a longitudinal length.

27. The catheter of claim 24, wherein said transition means is a metal plate that become thinner and more flexible along a longitudinal length.

28. The catheter of claim 17, wherein said transition means is a U-shaped wire sleeve.

29. The catheter of claim 28, wherein said U-shaped wire sleeve has a tight pitch at a first end and a loose pitch at a second end.

30. The catheter of claim 17, wherein said transition section is an extension of said distal shaft, such that said transition section is defined by the length of said transition means.

31. The catheter of claim 17, wherein said transition section is an extension of said proximal shaft after a point where said reinforcing means terminates, such that said transition section is defined from a point where said reinforcing means terminates to where said distal shaft begins.

32. The catheter of claim 17, wherein said proximal shaft further includes a longitudinal cut along the length thereof to facilitate the removal of a guidewire from said guidewire lumen.

33. The catheter of claim 32, wherein said longitudinal cut extends distally into said transition section.
